(19) United States
(12) Patent Application Publication
(10) Pub. No.: US 2020/0289593 A1
(43) Pub. Date: Sep. 17, 2020

(54) SPIRULINA CAROTENOID COMPLEX

(71) Applicant: WELLISEN INC., Reston, VA (US)

(72) Inventor: Jayant DESHPANDE, Edison, NJ (US)

(73) Assignee: WELLISEN INC.

(21) Appl. No.: 16/815,742

(22) Filed: Mar. 11, 2020

Related U.S. Application Data
(60) Provisional application No. 62/816,325, filed on Mar. 11, 2019.

(51) Int. Cl. A61K 36/02; A61K 9/00; A61K 31/015; A61K 31/047; A61K 31/202; A61K 33/30; A61K 33/34

(52) U.S. Cl. CPC A61K 36/02; A61K 9/0053; A61K 31/015; A61K 33/34; A61K 31/202; A61K 33/30; A61K 31/047

(57) ABSTRACT

Described herein are compositions comprising a spirulina complex enriched with carotenoids, gamma linolenic acid, zinc and copper. The extract is made highly bioavailable with the addition of suitable bioenhancers, which may also impart additional eye health benefits. The spirulina extract may be used as a dietary supplement or may be added to food or beverage product. Also provided herein is Als provided herein are methods for treating macular degeneration, dry eye syndrome and visual acuity comprising administering the compositions described herein to a patient in need of such treatment.

### BACKGROUND

Spirulina represents a biomass of cyanobacteria (blue-green algae) that can be consumed by humans and other animals. The two species are Arthrospira platensis and A. maxima.

The species A. maxima and A. plaetensis were once classified in the genus Spirulina. The common name, spirulina, refers to the dried biomass of A. platensis, which belongs to photosynthetic bacteria that cover the groups Cyanobacteria and Prochlorophyta.

Scientifically, a distinction exists between spirulina and the genus Arthrospira. Species of Arthrospira have been isolated from alkaline brackish and saline waters in tropical and subtropical regions. Among the various species included in the genus Arthrospira, A. platensis is the most widely distributed and is mainly found in Africa, but also in Asia. A. maxima is believed to be found in California and Mexico. The term spirulina remains in use for historical reasons.

Arthrospira species are free-floating, filamentous cyanobacteria characterized by cylindrical, multicellular trichomes in an open left-handed helix. They occur naturally in tropical and subtropical lakes with high pH and high concentrations of carbonate and bicarbonate. A. platensis occurs in Africa, Asia, and South America, whereas A. maxima is confined to Central America. Most cultivated spirulina is produced in open-channel raceway ponds, with paddle wheels used to agitate the water.

Provided in its typical supplement form as a dried powder, a 100-g amount of spirulina supplies 290 Calories and is a rich source (20% or more of the Daily Value, DV) of numerous essential nutrients, particularly protein, B vitamins (thiamin and riboflavin, 207% and 306% DV, respectively), and dietary minerals, such as iron (219% DV) and manganese (90% DV). The lipid content of spirulina is 8% by weight providing the fatty acids, gamma-linolenic acid, alpha-linolenic acid, linoleic acid, stearidonic acid, eicosapentaenoic acid (EPA), docosahexaenoic acid (DHA), and arachidonic acid. In contrast to those 2003 estimates (of DHA and EPA each at 2 to 3% of total fatty acids), 2015 research indicated that spirulina products “contained no detectable omega-3 fatty acids” (less than 0.1%, including DHA and EPA). An in vitro study reported that different strains of microalgae produced DHA and EPA in substantial amounts.

Spirulina generally has 300-700 mg/100 g of total carotenoids, of which 100-300 mg/100 g is beta carotene and 100-250 mg/100 g of zeaxanthin. It has 0.75 to 2% of Gamma Linolenic acid. Among minerals, zinc is present in the range of 1-3 mg/100 g and copper at 0.1-0.5 mg/100 g. To derive a significant eye health benefit such as overcoming macular degeneration, or dry eye syndrome, one needs to consume 15-20 gms of the powder. The bioavailability of the carotenoids and GLA from a spray dried powder of spirulina being low, such dose is unlikely to provide protection against macular degeneration or dry eye syndrome.

Extraction of carotenoids from spirulina is known in the art. While there are many publications in the literature that describe extraction of beta carotene, zeaxanthin, gamma linolenic acid, the yields are still very low and the ratios of carotenoids and fatty acids are not proper to elicit health benefits in the eyes. Moreover, none of the art-known compositions combine carotenoids with zinc and copper, which are essential in promoting eye health.

While beta carotene serves as a precursor for vitamin A biosynthesis, zeaxanthin gets deposited in macula and helps protect retina from light and free radicals. Zinc and copper are minerals essential for the smooth functioning of visual processes. Zinc is an essential trace mineral, or “helper molecule.” It plays a vital role in bringing vitamin A from the liver to the retina in order to produce melanin, a protective pigment in the eyes. Zinc is highly concentrated in the eye, mostly in the retina and choroid, the vascular tissue layer under the retina. Zinc supplementation has been known to interfere with copper absorption, so it is strongly recommended that people taking zinc also take 2 mg/day of copper. Gamma linolenic acid (GLA) which has strong anti-inflammatory action in the eyes, has been shown to increase lactoferrin production, helping to reduce dry eye syndrome.

Spirulina has several constituents that are useful in imparting eye health benefits such as overcoming macular degeneration, dry eye syndrome, improving visual processing speed, visual acuity, etc. However, these components occur in small concentrations and are hardly absorbed from the spray dried algal powder to elicit significant eye health benefit. Moreover, due to their chemical nature and polarity, it is generally considered to be impossible to have all these components in one extract of spirulina. Commercially available aqueous extract of spirulina may contain some vitamins and minerals, but they are devoid of carotenoids such as beta carotene and zeaxanthins. Omega-6 fatty acid, such as gamma linolenic acid is not part of the aqueous extract.

Efforts have been made to provide lepidic extract of spirulina, which are rich in carotenoids and gamma linolenic acid, but lack polar constituents that deliver significant benefit to the eyes. The phycocyanine extract, though rich in proteins, is poor in carotenoids and fatty acids.

Spirulina spray dried powder generally has about 1-1.5% of gamma linolenic acid, 0.1-0.5% of total carotenoids (of which 0.1-0.2% is beta carotene and 0.1-0.2% of zeaxanthin. It has 1-3 mg of zinc/100 g and 0.2-0.4 mg of copper/100 g. If one considers 5 g/day as a standard dose, the quantities of these eye health contributors one may derive are 50-75 mg of gamma linolenic acid, 5-10 mg of beta carotene/zeaxanthin, 50-150 micrograms of zinc and traces of copper. A fraction of these quantities may finally reach the blood stream due to several barriers for absorption. With these small quantities of phytoconstituents, one may not achieve the true eye health benefits. As an anti-inflammatory agent during dry eye syndrome, the dose of gamma linolenic acid may range between 2 and 2.5 grams/day. Beta carotene dose for eye health benefit ranges between 30-300 mg/day and zeaxanthin, without lutein, is needed in the dose levels of 6-30 mg/day. To prevent age related macular degeneration, body needs as high as 80 mg of zinc and 2 mg of copper. There is no available extract of spirulina that can provide these active principles in adequate quantities.

### SUMMARY

Provided herein is an extract of spirulina which contain all the components necessary to maximize eye health benefits in adequate quantities. The spirulina composition described herein solves the problems associated with prior art compositions, providing these active principles in their required concentrations in one extract. In addition, the process of extraction results in a composition with high bioavailability of these active principles for maximum benefits to eye health.

In particular, the compositions describe herein provide maximum benefits in terms of preventing macular degeneration, dry eye syndrome and loss of visual acuity. These compositions comprise beta carotene, zeaxanthin, gamma linolenic acid, zinc and copper. Also provided herein are methods of preventing and/or treating macular degeneration, dry eye syndronme, and/or loss of visual acuity comprising administering the compositions described herein to a patien in need of such treatment.

### DETAILED DESCRIPTION

Unless defined otherwise, all technical and scientific terms used herein have the same meaning as is commonly understood by one of skill in the art. All patents and publications referred to herein are incorporated by reference.

As used in the specification and claims, the singular form “a,” “an,” and “the” include plural references unless the context clearly dictates otherwise.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

The terms “treatment,” “treating,” “palliating,” and “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal so that both agents and/or their metabolites are present in the animal at the same time. Co-administration includes simultaneous administration in separate compositions, administration at different times in separate compositions, or administration in a composition in which both agents are present.

A “pharmaceutically acceptable salt” means a salt composition that is generally considered to have the desired pharmacological activity, is considered to be safe, non toxic and is acceptable for veterinary and human pharmaceutical applications. Pharmaceutically acceptable salts may be derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions described herein is contemplated. Supplementary active ingredients can also be incorporated into the compositions.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. Although antagonists herein generally interact specifically with (e.g. specifically bind to) the target, compounds that inhibit a biological activity of the target protein by interacting with other members of the signal transduction pathway of which the target protein is a member are also specifically included within the definition of “antagonist.”

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. Agonists herein generally interact specifically with (e.g. specifically bind to) the target, compounds that initiate or enhance a biological activity of the target polypeptide by interacting with other members of the signal transduction pathway of which the target polypeptide is a member are also specifically included within the definition of “agonist.”

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize the limits to the structural nature of the agents described herein.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” as applied to a biologically active agent refers to the agent's ability to selectively reduce the target signaling activity as compared to off-target signaling activity, via direct or interact interaction with the target.

“Subject” refers to an animal, such as a mammal, for example a human. The methods described herein can be useful in both human therapeutics and veterinary applications. In some embodiments, the patient is a mammal, and in some embodiments, the patient is human.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

Following Table explains the content of vital nutrients for eyes present in commercial spirulina powder, desired amounts of these constituents considering the poor bioavailability and quantities as per the instant invention.

| | | | Desired composition that | | | |
|---|---|---|---|---|---|---|
| Constituents | Composition of | | alleviates macular | | Composition as per | |
| essential for | commercial spray | | degeneration/dry eye | | the instant | |
| eye health | dried Spirulina at 5 | | syndrome at 5 gm/day | | invention at 5 | |
| benefit | gm/day dose | | dose | | gm/day dose @ | |
| Beta Carotene | 5-10 | mg | 30 | mg | 150-300 | mg |
| Zeaxanthin | 5-10 | mg | 24 | mg | 30-150 | mg |
| Gamma | 50-75 | mg | 2000 | mg | 2500-3000 | mg |
| Linolenic acid | | | | | | |
| Zinc | 50-150 | micrograms | 80 | mg | 100-120 | mg |

| | | | | | |
|---|---|---|---|---|---|
| Copper | traces | 2 | mg | 2-4 | mg |

Therefore, the instant invention provides several folds higher quantities of nutrients needed for eye health compared to standard commercial spirulina powder. The concentration of these nutrients in the composition may be from about 30 g/kg to about 60 g/kg of beta carotene; from about 6 g/kg to about 30 g/kg of zeaxanthin; from about 500 g/kg to about 600 g/kg of gamma linolenic acid; from about 20 g/kg to about 24 g/kg of zinc; and from about 400 mg/kg to about 800 mg/kg of copper.

Also described herein is a solid pharmaceutical composition for oral administration containing an effective amount of the spirulina extract described herein. In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Also described herein are anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, microcrystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions described herein to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teradecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, suitable lipophilic surfactants include, but are not limited to, glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the active ingredients described herein and to minimize their precipitation. A solubilizer may also be added to increase the solubility of hydrophilic active ingredients and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, [symbol] -caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, [symbol] -caprolactone and isomers thereof, [symbol] -valerolactone and isomers thereof, [symbol] -butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Suitable solubilizers include, but are not limited to, sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Also provided herein is a method of making the composition described herein, comprising preparing an extract of spirulina comprising therapeutic amounts beta carotene, zeaxanthin, gamma linolenic acid, zinc, and copper. In one of the embodiments, the extraction process used may be with standard solvents, but may involve selective/sequential extraction with series of polar/semipolar/nonpolar solvents. Alternatively, extraction with supercritical carbon dioxide in combination with solvent extraction may be employed. In a further alternative process, extraction with semipolar/non-polar solvents may be combined with partitioning with polar solvent. Regardless of In yet another embodiment, the extraction is followed by spray drying with a food grade carrier (1-35%).

In yet another embodiment, the extract is stabilized with food grade antioxidants (1-3%), chelating agents (0.5-1%), surfactants (1-3%) bioenhancers 1-5%) and pH modifiers (1-3%).

In yet another embodiment, the raw material used for the extraction may be a spent biomass from phycocyanin extraction process.

In yet another embodiment, the extract may contain additional fats/short and medium chain fatty acids (1-10%) that may support eye health benefits of spirulina.

### FOR IDS

U.S. Pat. No. 4,320,050 extraction of beta carotene and glycosided xanthophylls from spirulina.
H. Hadiyanto, Jurnal Teknologi, 77:1 (2015) 219-222
Zang Meiping, RRJBS/Phytopathology/Genes & Diseases—S1, 2015
Lourdes Casas Cardoso, American Journal of Analytical Chemistry, 2012, 3, 877-883
Bolan Yu, British Journal of Nutrition, (2012), 108, 611-619.
Lei, Li, J Nutr Sci. 2012; 1: e19.
Ratana Chaiklahana, ScienceAsia 34 (2008): 299-305.

What is claimed is:

1. A spirulina extract composition comprising beta carotene, zeaxthanin, gamma linolenic acid, zinc, and copper.

2. The composition of claim 1, comprising about 30 g/kg to about 60 g/kg of beta carotene; about 6 g/kg to about 30 g/kg zeaxanthin; about 500 g/kg to about 600 g/kg of gamma linolenic acid; from about 20 g/kg to about 24 g/kg of zinc; and from about 400 mg/kg to about 800 mg/kg of copper.

3. The composition of claim 1, which is prepared by extracting the beta carotene, zeaxthanin, gamma linolenic acid, zinc, and copper from spirulina by a process comprising solvent extraction.

4. The composition of claim 3, wherein the solvent extraction process comprises one or more steps of elective/sequential extraction with series of polar/semipolar/nonpolar solvents.

5. The composition of claim 3, wherein the process further comprises supercritical carbon dioxide extraction.

6. The composition of claim 3, wherein the process further comprises a step of partitioning with polar solvent.

7. The composition of claim 3, wherein the process further comprises a step of spray drying with a food grade carrier

8. The composition of claim 7, wherein the concentration of the food grade carrier is from about 1% to about 35%.

9. The composition of claim 1, further comprising one or more stabilizers selected from the group consisting of food grade antioxidants, chelating agents, surfactants, bioenhancers, and pH modifiers.

10. The composition of claim 9, wherein concentration of the food grade antioxidants in the composition is about 1% to about 3%.

11. The composition of claim 9, wherein the concentration of the chelating agents in the composition is about 0.5% to about 1%.

12. The composition of claim 9, wherein the concentration of surfactants in the composition is about 1% to about 3%.

13. The composition of claim 9, wherein the the concentration of bioenhancers in the composition is about 1% to about 5%.

14. The composition of claim 9, wherein the concentration of pH modifiers in the composition is about 1% to about 3%.

15. The composition of claim 3, wherein the spirulina comprises spent biomass from a phycocyanin extraction process.

16. The composition of claim 1, further comprising fats and/or short and medium chain fatty acids.

17. The composition of claim 16, wherein the concentration of fats and/or short and medium chain fatty acids in the composition is from about 1% to about 10%.

18. A method of treating macular degeneration and/or dry eye syndrome in a patient in need of such treatment, comprising administering to the patient an effective amount of a composition according to claim 1.

19. The method of claim 18, wherein the route of administration is oral.

20. The method of claim 18, wherein the composition is administered to the patient in an amount of 5 g/day.
